(19) United States
(12) Patent Application Publication
(10) Pub. No.: US 2009/0023144 A1
(43) Pub. Date: Jan. 22, 2009

(54) METHOD AND ITS KIT FOR QUANTITATIVELY DETECTING SPECIFIC ANALYTE WITH SINGLE CAPTURING AGENT

(75) Inventor: Dongxu Sun, Jiangsu (CN)

Correspondence Address: SCHMEISER, OLSEN & WATTS, 22 CENTURY HILL DRIVE, SUITE 302, LATHAM, NY, 12110, US

(73) Assignee: XUZHOU LINGXIN BIOSCIENCES INC., Jiangsu (CN)

(21) Appl. No.: 11/915,616

(22) PCT Filed: May 25, 2006
(86) PCT No.: PCT/CN2006/001099

§ 371 Date: Nov. 27, 2007

(30) Foreign Application Priority Data
May 30, 2005 (CN) 200510040295.6

(51) Int. Cl. G01N 33/543; C12Q 1/68

(52) U.S. Cl. 435/6; 436/518; 435/7.92; 436/527; 436/528; 436/530

(57) ABSTRACT

The invention provides a method and its kit for quantitatively detecting a specific analyte with a single capturing agent. The quantitative detection of a specific analyte with a single capturing agent comprises: firstly combining the tested analyte with a solid phase capturing agent, then labeling analyte which has been trapped by the capturing agent with a report molecule; secondly eluting the labeled analyte from the complex, recombining the tested analyte with a new solid phase capturing agent, and ascertaining the content of analyte by detecting the report molecule's label signal. The kit of the invention comprises a capturing device, a detecting device, a report molecule for labeling and an analysis substance eluate. The advantages of the invention are the need of one single capturing agent, the capability of detecting for many analytes which can't be tested at present, wide application, high sensibility and low noise. The invention can be applied to diagnosis, medical expertise, new medicine development, application of protein micro array and chip, and fundamental research.

### TECHNICAL FIELD

This invention relates to the field of biotechnology; particularlly, it relates to a new method of using single capturing reagents to quantitatively detect specific analytes, and to reagent kits based on the method.

### BACKGROUND TECHNOLOGIES

Measuring a specific protein factor(s) in a biological (including human) sample is of great importance in applications and basic researches in medical, biological, agricultural and environment protection fields. For example, detection of specific protein components of pathogenic microorganisms is currently an important tool for the diagnosis of infectious diseases. Similarly, measurement of early specific protein biomarkers in cancers and cardiovascular diseases is extremely important for early diagnosis, early treatment and monitoring treatment efficacy of these diseases. Depending on the purpose of the applications, the analytes to be tested may be a single protein or a few protein factors, or may be a group of proteins of different numbers. The recent rapid advancement in genomic and proteomic researches and applications sends a strong demand for multiplexed assays of hundreds or thousands of proteins or even the entire proteome in a biological sample simultaneously.

Facing this increasing demand, various methods for protein detection and proteomics researches have been developed in recent years. These include various types of immunoassays, 2-dimentional gel electrophoresis, mass spectrometry and peptide spectrometry, etc. Among them, the most conveniently and widely used method is the enzyme-linked immunosorbent assays (ELISAs). The typical ELISA method utilizes two different antibodies recognizing the same antigen molecule at different epitopes. The two antibodies should be coordinately paired, i.e., binding of one antibody to the antigen should not interfere with the binding of the other antibody to the same antigen molecule. This type of ELISAs, commonly referred to as Sandwich ELISA, include the following key steps: (1) coating a capture antibody onto a solid surface, usually the interior surface of wells of a microtiter plate; (2) adding the sample to be tested into the wells and letting the analyte (the antigen) in the sample bind specifically to the capture antibody and then removing the unbound materials; (3) adding a detection antibody that has been labeled with some kind of reporter molecules (enzymes, biotin, fluorescent groups such as fluorescein, or other types of molecules), letting the detection antibody bind specifically to the captured analyte and then removing the unbound detection antibody; (4) adding relevant reagents necessary for the reporter molecules to generat assay signals (e.g., enzyme-labeled streptavidin, enzyme substrate, etc), or directly detecting the presence of the reporter molecules. The concentration of the analyte in the sample can be calculated by comparing the signal generated from the sample and those from known standards of the same analyte. While Sandwich ELISAs are usually specific and sensitive (usually the sensitivity is at or below 0.5-2 ng/ml), this method has three major limitations. First, to develop a sandwich ELISA for an analyte, it is necessary to have two antibodies (the capture antibody and the detection antibody) against the same analyte; both antibodies should possess high specificity and affinity to the analyte. Moreover, they should be coordinately paired to each other, i.e., the binding of the capture antibody to the analyte does not interfere with the binding of the detection antibody to the same analyte molecule. These requirements have, to a large extent, limited the broader application of the Sandwich ELISA. This is because it usually takes a great effort and a long time to develop such antibody pairs against the same analyte. For proteins (or protein domains) of low molecular weights or with just a few antigenic epitopes, it is even harder to establishing such coordinately paired antibodies. Second, the Sandwich ELISA usually requires the labeling of each species of detection antibody with a reporter molecule. If the goal of the test is to quantitatively assay hundreds or even thousands of proteins, every detection antibody has to be labeled separately,. This is not only a huge and tedious task, but also the labeling of antibodies may have different efficiency in different lots for each antibody and among different antibodies, causing variations in the detection. In addition, the chemical modification of the detection antibodies by the labeling process may also affect the antibody-antigen binding. Third, in performing multiplexed assays, the Sandwich ELISA requires mixing all the labeled detection antibodies together, thus greatly diluting each individual antibody, decreasing the assay signals and increasing nonspecific binding and the background noise. These limitations prevent broader use of the Sandwich ELISA, particularly making it difficult to use Sandwich ELISA as a major technical platform in the proteomic (for example, using protein microarrays) studies.

To overcome the limitations of sandwich ELISA described above, a number of improvements have been proposed and implemented. One of them involves pre-labeling all the proteins in a biological sample with a reporter molecule such as a fluorescent dye (Miller et al. 2003. Proteomics. 3:56-63). The pre-labeled sample is then added to a solid phase coated with a capture antibody (antibodies). After removing nonspecific materials, the bound analyte can be measured by directly detecting the signal derived from the reporter molecules bound to the antibodies on the solid phase. This method is relatively easy and straightforward, requiring just one antibody (the capture antibody) to detect one analyte, but it has the following disadvantage. First, there are frequently thousands types of molecules of different natures and sizes in a biological sample, and many of them may directly or indirectly interfere with the pre-labeling of the specific analyte in question. Proteins present at low concentrations may not be labeled efficiently. Second, the process of pre-labeling may modify the specific antigenic determinant region on protein molecules, lowering or even completely abolishing its ability to bind to the specific capture antibody on the solid phase. It has been shown that assays based on the analyte pre-labeling method usually have low sensitivity and high background noise.

In order to increase the detection sensitivity of Sandwich ELISA, immuno-PCR has been proposed, in which a DNA oligonucleotide is used to label the detect antibody. The signal is then amplified and recorded by PCR or rolling-circle replication. Although these methods can significantly increase the assay sensitivity, the experimental procedures are rather complicated and tedious, and the cost is high. More importantly, the limitations of the Sandwich ELISAs mentioned earlier are still present with the immuno-PCR method.

### SUMMARY OF THE INVENTION

To overcome the limitations of the currently widely used immunoassay methods, this invention describes a new assay method of using just one capturing reagent to quantitatively, sensitively and conveniently detect an analyte. This method is called Specific Analyte Labeling and Recapture Assay, abbreviated as SALRA. The principle of this method is as the following: the analyte captured by the capturing reagent is labeled with reporter molecules; the labeled analyte is eluted from the complex and recaptured by a new capture reagent on a solid phase; the concentration of the analyte is determined by detecting the signals derived from the labeled reporter molecules. The SALRA method can be applied to various types of solid-phase based assay platforms such as microtiter plates, filter membranes, protein (antibody) microarray chips, micro-magnetic beads, etc. It can be used to detect one or several analyte(s) at a time, and it can also be applied to multiplexed detection of tens, hundreds or even thousands of different analytes at the same time. The method will mainly be used to detect the binding between antibodies and antigens, but it can also be used to detect other types of protein-protein binding or binding between proteins and other types of molecules. In addition, the SALRA method can also be used to facilitate identification of hybridoma clones producing specific monoclonal antibodies. Furthermore, this invention also proposes and describes detection kits based on the SALRA method of using single capture reagents to quantitatively measure specific analytes.

This invention describes a method of using single capturing reagents to quantitatively measure analytes, wherein the characteristics are:

(1) Capture the analyte. A capturing reagent is used to coat a solid surface to form the “Capture Device”. The biological sample to be tested is added to the Capture Device and the analyte in the sample is allowed to be captured by the capturing reagent, thus forming the analyte-capturing reagent complex.

(2) Label the analyte. A reporter molecule is used to label the analyte-capturing reagent complex.

(3) Elute the analyte. The labeled analyte is eluted from the complex.

(4) Recapture. The eluted analyte is properly neutralized and diluted, and allowed to bind to the capturing reagent on the Detection Device, wherein said Detection Device is a solid surface coated with the capture reagent.

(5) Detection. The unbound materials are removed from the Detection Device, and the concentration of the analyte is determined by detecting the intensity of the signal derived from the reporter molecules.

In the invention described above, said capturing reagent may be an antibody, fragments of an antibody, a non-antibody protein, a peptide, an oligonucleotide or a small molecule. When the capturing reagent is an antibody, said antibody is preferentially a monoclonal antibody.

In the invention described above, said analyte may be a protein antigen, an antibody, a protein of other types, a peptide, an oligonucleotide apatamer, a member of other types of biological macromolecules, a complex of different biological molecules, a small molecule compound, or a subcellular structure, etc., that can be specifically captured by the capturing reagent.

In the invention described above, said reporter molecules includes, but not restricted to, biotin, fluorescein or other fluorescent compounds, enzymes, peptides and oligonucleotides.

The details of the SALRA method are provided below using antibody-antigen as an example.

(1) A monoclonal antibody is used to coat the surface of a solid device, such as the surface of wells of a microtiter plate, a nylon (or other material) filter membrane or magnetic beads, etc. This solid surface is used to capture the specific antigen in a biological sample, and is called the Capture Device. Depending on the purpose of the assay and the nature of the sample(s), the coating material may be a single antibody, or may be a mixture of a number of different antibodies against different antigens (for multiplexed detection of multiple antigens at the same time). After the coating process, the unbound areas of the solid surface of the Capture Device are blocked with an excess amount of nonspecific proteins (such as non-fat milk or bovine serum albumin). The blocking solution is removed and washed afterwards.

The biological sample to be tested is added to the Capture Device to allow the binding and capture of the specific antigen in the sample by the antibody on the Capture Device to form the tightly bound antigen-antibody complex. Unbound proteins and other materials are removed and washed from the Capture Device.

(2) The antigen-antibody complexes are labeled with a reporter molecule, for example, by adding small molecular compounds that can covalently modify the side chains of proteins. These compound carry certain reporter molecules (biotin, fluorescein, etc.), so that the reporter molecules can be conjugated to the surface of the antigen molecules bound to the antibodies on the Capture Device. For example, certain N-hydroxysuccinimide (NHS) based compounds, such as NHS-biotin, NHS-fluorescein, NHS-peptide, NHS-oligonucleotide, can covalently conjugate the reporters they carry (biotin, fluorescein, peptide, oligonucleotide, etc) to the primary amine of the lysine residues in proteins. Taking NHS-biotin as an example, because primary amines are almost universally present in all proteins, NHS-biotin can practically label almost all proteins. It should be noted that, since the epitopic region of the antigen is bound to the antibody in the antigen-antibody complex, it is protected from the labeling process by NHS-biotin, therefore still keeping the ability to bind to the same antibody after it is eluted from the complex. Besides biotin, other reporter can also be used, depending on the detection systems and the goal of the assay. Examples of other reporters include oligonucleotides and fluorescent dyes (such as fluorescein). Fluorescent dyes as reporters are particularly useful in protein microarrray-based detection. In addition to NHS, other active chemical groups may also be used to covalently attach the reporter molecules to other side chain groups (such as sulfhydryl, carboxyl or hydroxyl groups) of proteins.

(3) A solution containing an excess amount primary amines, such as a Tris-HCl buffer, is added to quench and remove the free NHS-biotin molecules that have not been covalently linked to proteins. Then a small volume of an elution solution is added to dissociate the labeled antigen from the antibody. For example, 0.1M citric acid (pH2.8) or commercially available elution solutions for dissociating antigen-antibody complexes (such as the ImmunoPure Elution buffer from PIERCE, USA), can be used. The eluant that contains the labeled antigen is transferred from the Capture Device, neutralized and diluted by adding 3-10 volumes of an appropriate buffer (containing nonspecific proteins such as non-fat milk). The purpose of the neutralization step is to restore the pH to near neutral and lower the concentration of the elution solution so that the binding between the labeled antigen and the same species of the antibody is not inhibited.

(4) The neutralized, reporter-labeled antigen is added to the Detection Device. Depending on the systems and purposes of the assays, the Detection Device can be made of a microtiter plate, a filter membrane, magnetic beads or a planer surface of plastic or glass (protein chips). The solid surface of the Detection Device is coated with the same type of antibodies used for coating the Capture Device, and has already been blocked with nonspecific proteins. However, if the antibodies on the capture Device are a mixture of a number of different antibodies for different antigens, each of these antibodies are spatially separated from each other on the Detection Device and not mixed. When the Detection Device is a microtiter plate, each well is coated with one antibody only. If the Detection Device is a protein chip, the antibodies are individually spotted in a microarray format, with each antibody occupying a unique geographic location. The Detection Device should be prepared and blocked in advance in a timely manner, so that the labeled antigen from the Capture Device can be added immediately after the elution and neutralization.

(5) On the Detection Device, the labeled antigen is recaptured by the corresponding antibody. After removing unbound materials, the signal can be developed and recorded. For example, if the reporter molecule is biotin, the signal can be generated by adding an enzyme (usually horse radish peroxudase or alkaline phosphatase)-conjugated avidin or streptavidin. Biotin and avidin (streptavidin) have very high affinity and specificity to each other, so the enzyme conjugated to avidin (streptavidin) can be stably attached to the surface of the recaptured antigen molecule. After removing unbound enzyme avidin (streptavidin) conjugates, the enzyme substrate is added to produce a colorimetric, fluorescent or luminescent signal, which can be readily recorded by reading the plate or scanning the fluorescence on the Detection Device. If a fluorescent dye is used as the reporter, the signal can be directly measured by reading or scanning the intensity of the fluorescence. The concentration of the specific antigen in the sample can be calculated from these signals.

The contribution of this invention is that it provides a new method, the SALRA method, as illustrate in FIG. 1, wherein an analyte in a biological sample can be quantitatively assayed by using only a single capturing reagent. The individual steps and the detailed experimental techniques described in this invention, such as coating with capturing reagents, binding of analytes to capturing reagents, developing and measuring the signals derived from the reporter molecules, are familiar to those experienced in this field.

Based on this invention, commercial detection kits can be developed, wherein said kits include Capture Devices, Detection Devices, the reporter chemicals for labeling the analytes, reagents required for eluting the analyte, etc.

Obviously, the Specific Analyte Labeling and Recapture Assay (SALRA) method described in this invention for quantitatively measuring analytes by using a single capturing reagent, can be used in clinic diagnosis, identification and detection of biomarkers, proteomic researches, new drug target identification, pharmacokinetic and pharmacodynamic analysis, etc.

Comparing to the widely used traditional Sandwich ELISA method, the SALRA method described in this invention has the following advantages:

(1) The SALRA method requires only a single capturing reagent to quantitatively detect an analyte, i.e., requiring only a single antibody to detect an antigen, while the Sandwich ELISA method requires two paired antibodies for the same purpose. Obviously, developing one monoclonal antibody is much easier than developing two monoclonal antibodies that can form an ELISA pair. Therefore, for many proteins for which at present there is still no ELISA available, the SALRA method can immediately provide quantitative assays. Furthermore, the SALRA method can be used to detect proteins or protein functional domains that carry just one epitopes or with a few epitopes that are very closely adjacent to each other, such as phosphorylated motifs of proteins, important functional domains or their activated status, small peptides, specific oligonucleotides or certain small organic compounds, etc. Therefore, the SALRA method has a wide range of applications, and should facilitate proteomic researches, clinical diagnosis, drug discovery, safety inspections of food and agricultural products, and environmental protection.

(2) The SALRA method does not require the labeling of antibodies in advance. No matter how many proteins are assayed at the same time, the method requires only one labeling small molecule for the analytes.

(3) High specificity and low background noise. There are two steps in the SALRA method to safeguard assay specificity and reduce the background noise. First, the Capture Device captures the specific analyte in the sample. When the analyte is being labeled, most of nonspecific materials are already removed. Therefore, instead of total proteins and all other materials in the sample, only the analytes captured by the capturing reagents can be labeled. Second, in the process of recapturing the labeled analyte on the Detection Device, nonspecific materials are further removed by washing steps so that they have no place to stay on the Detetion Device.

(4) High sensitivity. The step of labeling the analytes in the SALRA method is also an important step of signal amplification. For example, when NHS-biotin is used to label the captured antigen, because of the presence of at least several or even tens of lysine residues in most of proteins, each protein molecule can be labeled with several or even tens of biotin moieties, resulting in increased signal intensity and increased detection sensitivity. In addition, the foregoing described 2 steps that ensure assay specificity also provide rooms to increase assay sensitivity: because of the low background noise, milder washing conditions can be used to increase the binding between antibodies and antigens. This is particularly important for antibody-antigen pairs that have relatively low affinities. Moreover, because the Capture Device and the Detection Device are separated, the analyte captured on the Capture Device can be properly concentrated before adding to the Detection Device to increase the assay sensitivity. For example, wells of larger surface areas of a microtiter plate can be used to make the Capture Device to increase the surface area for analyte capturing, meanwhile the surface area of the Detection Device can be reduced to make it possible to increase the concentration of the analyte on the Detection Device.

(5) The SALRA method is particularly useful for multiplexed assays of many proteins at the same time. In multiplexed assays, many analytes can be measured by coating the Capture Device with a mixture of multiple capturing reagents and individually spotting the capturing reagents on the Detection Device. The SALRA method makes it possible to perform multiplexed assays with small quantities of biological samples. It is particularly suitable for multiplexed assays and proteomic analysis of small samples (such as biopsy samples).

(6) The principle and methodology of SALRA can also be suitable for detecting protein-protein interactions that are not of antibody-antigen nature, or interactions between proteins and other types of molecules. Such molecular interactions are very important in studying various cellular processes, diagnosing disease progressions, and developing new drugs, etc. For example, in order to test the level of a protein factor (protein X) in a biological sample, another protein (protein Y) to which protein X binds can be used as the capturing reagent to prepare the Capture Device and the Detection Device. Following the SALRA procedures, the sample is added to the Capture Device and the protein X is captured and labeled. The labeled protein X is eluted and recaptured on the Detection Device, and detection of protein X can be achieved thereafter. Besides proteins, other types of molecules, such as peptides, nucleic acids, polysaccharides, lipids, and even small molecules, can also be used as capturing reagents to detect various types of analytes that they specifically bind to, including proteins, peptides, oligonucleotide aptamers, small molecules, etc.

### FIGURE LEGENDS

FIG. 1. The scheme of the steps of this invention

FIG. 2. The assay results of Specific Example 1.

FIG. 3. The assay results of Specific Example 2.

### SPECIFIC EXAMPLES

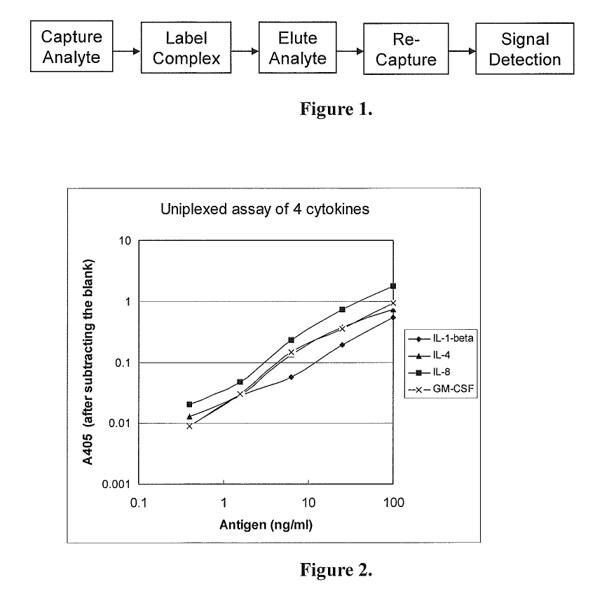

The following specific examples are provided to describe the implementation of the SALRA method as illustrated in FIG. 1 in further details.

### Specific Example 1

### Uniplexed Assay (Detecting One Protein)

In this Specific Example, the Capture Device and the Detection Device were both made of 96-well microtiter plates. The Capture Device was coated with a single antibody for detecting one antigen. The analytes were 4 human cytokines: IL-1-beta, IL-4, IL-8 and GM-CSF. Their corresponding antibodies were all monoclonal antibodies from Biolegend, USA).

(1) Capturing the Antigens

a. Coating with the capture antibodies: Two 96-well microtiter plates (flat-bottom, with high binding capacity to proteins) were used, one (the Capture Device) for capturing the antigens in samples, and the other (the Detection Device) for detecting the labeled antigens. To each well, 100 μl of a capture monoclonal antibody at 0.5 μg/ml (diluted in phosphate-buffered saline, PBS) was added. On each plate, 8 wells were coated with each of the monoclonal antibodies against IL-1-beta, IL-4, IL-8 and GM-CSF. The plates were incubated at 4° C. overnight.

b. Blocking nonspecific binding sites. The unbound antibodies were removed from the wells, and the wells were washed once with PBS containing 0.1% Tween 20 (PBST) and blocked with 400 μl of 2% non-fat milk dissolved in PBS at room temperature. The Capture Device was blocked for 1 hour, and the Detection Device was blocked until prior to use (about 4 hours).

c. Capturing the antigens. The blocking solution was removed from the Capture Device. To each well coated with the corresponding antibody, 100 μl of each of the 4 cytokine at different concentrations (diluted in PBS+1% non-fat milk) was added. The plate was incubated at room temperature for 1.5 hours.

(2) Labeling the Antigens

The unbound antigens and non-specific materials were removed from the Capture Device and the wells were washed twice with PBST and once with PBS. To each well, 100 μl of 0.02% NHS-biotin (dissolved in PBS) was added and the plate was incubated at room temperature for 30 minutes. The unincorporated NHS-biotin was then quenched and washed by adding 10 mM Tris-HCl, pH8.0 (5 minutes at room temperature). The Tris-HCl buffer was then removed.

(3) Eluting the Antigens

To each well containing the labeled antigens, 20 μl of the Elution Buffer (the ImmunoPure Elution Buffer from PIERCE, USA) was added and the plate was incubated at room temperature for 15 minutes. At the same time, the blocking solution in the Detection Device was removed and 180 μl of PBS+1% non-fat milk was added.

(4) Recapturing the Antigens

The entire content (about 20 μl) of each well from the Capture Device was transferred to the wells of the Detection Device coated with the corresponding antibodies. Because the wells of the Detection Device already contained 180 μl of PBS+1% non-fat milk, the eluted antigen was neutralized and diluted 10-fold, so that the rebinding (recapture) of the labeled antigens to the specific antibodies on the Detection Device would not be inhibited.

(5) Detection

a. Developing the signal: Unbound materials were removed and the wells were washed twice with PBST and once with PBS. To each well, 100 μl of HRP-conjugated avidin (diluted to 1 μg/ml in PBS containing 1% non-fat milk) was added. The plate was incubated at room temperature for 30 minutes. The wells were washed three times with PBST and once with PBS. Then 100 μl of an HRP substrate solution (0.3 mg/ml ABTS, 0.02% H₂O₂) was added to each well. The plate was incubated at 37° C. for 30 minutes and the absorbance at 405 nm was recorded.

b. Results of the detection: FIG. 2 shows the results of the Specific Example 1. The intensity of signals of the four cytokines tested showed certain linear correlations to the concentrations of each antigen from 100 ng/ml to 0.4 ng/ml, indicating that the SALRA method can be used to detect these cytokines within this range of concentrations. The sensitivities of the assays were at least 0.4 ug/ml.

### Specific Example 2

### Multiplexed Assay (Detecting Three Proteins Simultaneously)

In this specific example, the Capture Device and the Detection Device were both made of a 96-well microtiter plates. The Capture Device was coated with a mixture of three antibodies for detection of three antigens simultaneously. The analytes to be tested were 3 human cytokines: IL-1-beta, TNF-alpha and IL-10. Their corresponding antibodies were all monoclonal antibodies.

(1) Capturing the Antigens

a. Coating with the capture antibodies: Two 96-well microtiter plates (flat-bottom, with high binding capacity to proteins) were used, one for capturing the antigen (the Capture Device) and another for detecting the signal (the Detection Device). The wells in the Capture Device were coated with 100 μl of a mixture of the 3 monoclonal antibodies against human IL-1-beta, TNF-alpha and IL-10, each at 0.5 μg/ml (in PBS). Eight wells were coated. The wells of the Detection Device were separately coated with just one of these three antibodies (at 0.5 μg/ml in PBS). For each antibody, 8 wells were coated. The plates were incubated at 4° C. overnight.

b. Blocking nonspecific binding sites. The procedure was the same as that described in Specific Example 1.

c. Capturing the antigen. The blocking solution was removed from the wells of the Capture Device. To each well, 100 μl of a mixture of the three cytokines to be tested at different concentrations (in PBS+1% non-fat milk) was added. The plate was incubated at room temperature for 1.5 hours. The concentrations of the three cytokines in each well were combined as in Table 1.

TABLE 1
The concentrations (ng/ml) of the three cytokines tested

| Well # | IL-1-beta | TNF-alpha | IL-10 |
|---|---|---|---|
| 1 | 100 | 0.1 | 6.25 |
| 2 | 25 | 0.025 | 1.6 |
| 3 | 6.25 | 0 | 0.4 |
| 4 | 1.6 | 100 | 0.1 |
| 5 | 0.4 | 25 | 0.025 |
| 6 | 0.1 | 6.25 | 0 |
| 7 | 0.025 | 1.6 | 100 |
| 8 | 0 | 0.4 | 25 |

(2) Labeling the Antigens

The procedure was the same as that described in Specific Example 1.

(3) Eluting the Antigens

To each well, 20 μl of the Elution Buffer (the same as that described in Specific Example 1) was added and the plate was incubated at room temperature for 15 minutes. At the same time, the blocking solution in the Detection Device was removed and 60 μl of PBS+1% non-fat milk was added to each well.

(4) Recapturing the Antigens

The antigens eluted from each well of the Capture Device was transferred to three wells of the Detection Device coated with different single antibodies, 6 μl into each well. Because the wells of the Detection Device already contained 60 μl of PBS+1% nonfat fat milk, the antigen elution solution was neutralized and diluted so that the rebinding (recapture) of the labeled antigens to the specific antibodies on the Detection Device would not be inhibited.

(5) Detection

a. Developing the signal: The procedure was the same as that described in Specific Example 1.

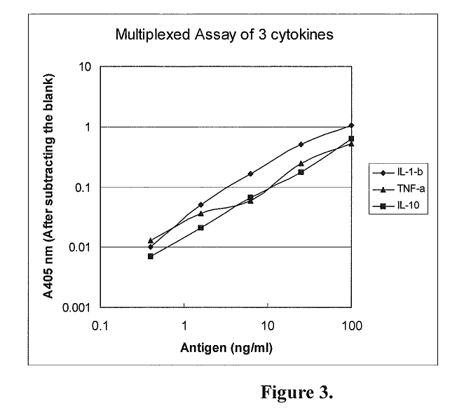

b. Results of the detection: FIG. 3 shows the results of the Specific Example 2. The intencity of signals of the three cytokines tested showed certain linear correlations to the concentrations of each antigen from 100 ng/ml to 0.4 ng/ml, indicating that the SALRA method can be used to simultaneously detect multiple proteins in a multiplexed assay. In this Specific Example, the sensitivity of each cytokine assays was at least 0.4 ug/ml.

1-8. (canceled)

9. A method using a single capturing reagent to quantitatively measure an analyte, wherein said method comprising the following steps:
(a). capture the analyte: coat a solid surface of a device with capturing reagent(s) to form a “capture device”; add a biological sample to be tested and let the said capturing reagent(s) bind to the specific analyte(s) in the sample to form a capturing reagent-analyte complex;
(b). label the complex: use a reporter molecule to label the capturing reagent-analyte complex;
(c). elute the analyte: separate the labeled analyte from the complex with an elution buffer;
(d). recapture: neutralize and dilute the eluted analyte and let the capturing reagent on a detection device bind to the labeled analyte, wherein said detection device is a solid surface coated with the same or different capturing reagent;
(e). detection: determine the level of said analyte by measuring the signal produced by said reporter molecule.

10. The quantitative assay method of claim 9, wherein said capturing reagent is an antibody, a fragment of an antibody, a non-antibody protein, a peptide, an oligonucleotide or a small molecule compound.

11. The quantitative assay method of claim 10, wherein said antibody is a monoclonal antibody.

12. The quantitative assay method of claim 9, wherein said analyte is a protein antigen, an antibody, a peptide, an oligonucleotide apatamer, other biological macromolecules or their complexes, or a subcellular structure, which can be specifically bound to said capturing reagent.

13. The quantitative assay method of claim 9, wherein said reporter molecule is biotin, fluorescein or other fluorescent functional groups, enzymes, peptides, or oligonucleotides.

14. The quantitative assay method of claim 10, wherein said reporter molecule is biotin, fluorescein or other fluorescent functional groups, enzymes, peptides, or oligonucleotides.

15. The quantitative assay method of claim 11, wherein said reporter molecule is biotin, fluorescein or other fluorescent functional groups, enzymes, peptides, or oligonucleotides.

16. The quantitative assay method of claim 12, wherein said reporter molecule is biotin, fluorescein or other fluorescent functional groups, enzymes, peptides, or oligonucleotides.

17. The quantitative assay method of claim 9, wherein said solid surface of a capture device is a test tube, a microtiter plate, filter membrane, detection paper, or micro-magnetic beads; wherein said solid surface of a detection device is a microtiter plate, filter membrane, detection paper, micro-magnetic beads; or planar thin carriers made of glass or plastic.

18. The quantitative assay method of claim 9 to be applied in clinical diagnosis, biomarker identification and analysis, proteomics researches and analysis, new drug target identification and validation, clinical pharmacokinetics and pharmacodynamics analyses.

19. Kits applying the method of claim 9 of using a single capturing agent to quantitatively measuring an analyte, wherein said kit including: capture device, detection device, reporter molecule(s) to be used for labeling the analyte, and elution solution for analyte elution, etc.
